(19) United States
(12) Patent Application Publication
(10) Pub. No.: US 2023/0390457 A1
(43) Pub. Date: Dec. 7, 2023

(54) DE-EPITHELIALIZED PLACENTAL TISSUE GRAFTS AND METHODS OF PREPARING AND USING THE SAME

(71) Applicant: SNOASIS MEDICAL LLC, Golden, CO (US)

(72) Inventors: Robert TOFE, Denver, CO (US); Peter MARINER, Superior, CO (US)

(73) Assignee: SNOASIS MEDICAL LLC, Golden, CO (US)

(21) Appl. No.: 18/032,099

(22) PCT Filed: Oct. 15, 2021
(86) PCT No.: PCT/US2021/055202

§ 371 Date: Apr. 14, 2023

Related U.S. Application Data
(60) Provisional application No. 63/093,083, filed on Oct. 16, 2020.

(51) Int. Cl. A61L 27/36; A61L 27/56

(52) U.S. Cl. CPC A61L 27/3604; A61L 27/56; A61L 27/3687; A61L 27/3691; A61L 2430/34; A61L 2430/40

(57) ABSTRACT

The present disclosure relates to compositions and methods for obtaining a modified amnion, a spongy intermediate layer, and a chorion from a placental membrane tissue by removing the amniotic epithelium layer to achieve a modified amnion wherein the modified amnion does not contain an amniotic epithelium layer and wherein the spongy intermediate layer is disposed between the modified amnion and the chorion.

### CROSS REFERENCE TO RELATED APPLICATIONS

This application claims the benefit of U.S. Provisional Application No. 63/093,083, filed Oct. 16, 2020, the disclosure of which is hereby incorporated by reference in its entirety.

### FIELD OF THE INVENTION

The present disclosure relates to compositions and methods for obtaining a modified amnion, a spongy intermediate layer, and a chorion from a placental membrane tissue by removing the amniotic epithelium layer to achieve a modified amnion wherein the modified amnion does not contain an amniotic epithelium layer and wherein the spongy intermediate layer is disposed between the modified amnion and the chorion.

### BACKGROUND OF THE INVENTION

The membranes of a human placenta can serve as a substrate material, more commonly referred to as a biological dressing or patch graft, which has various medical applications, including use in surgical, ophthalmic, dental, and wound healing procedures.

Human placental membranes derived from the amniotic sac consist of three main sections, an amnion, a spongy intermediate layer, and a chorion. The unique physical and biological properties of each of the three placental sections make them ideally suited for even the most challenging environments. Whereas the basement membranes provide barrier function and support for cellular layers, the compact and reticular layers provide elastic and tensile strength that allows the membrane to stretch and bend without failing. The fibroblast and trophoblast layers do not contribute significantly to physical strength of the membrane; instead, these layers contain most of the soluble factors that are attributed to these membranes, including antimicrobial peptides, growth factors, and cytokines. Accordingly, maintaining the physical and biological properties of these three sections is important for graph success.

Epithelial layers create a non-adhesive surface that limits cellular attachment. As such, surgical use of placental membranes requires specific orientation in or on a wound to function successfully. Further, the placental membranes must be cut to fit the treatment site without allowing the placenta membrane to fold over on itself, thereby creating an area/section of the membrane that is improperly oriented.

### SUMMARY OF THE INVENTION

An aspect of the present disclosure provides for compositions having a modified amnion, a spongy intermediate layer, and a chorion. In some embodiments, the modified amnion does not contain an amniotic epithelium layer. In some embodiments, the spongy intermediate layer can be disposed between the modified amnion and the chorion. In some aspects, the modified amnion can have a first side which is an exposed basement membrane and wherein the spongy intermediate layer can be disposed between the modified amnion and the chorion. In some aspects, the modified amnion can have a first side which is an exposed basement membrane that can be substantially free of epithelial cells, and wherein the spongy intermediate layer can be disposed between the modified amnion and the chorion. In some aspects, the modified amnion can further have a compact stromal layer, a fibroblast layer, or a combination thereof. In some embodiments, the chorion can have a basement membrane. In some aspects, the chorion can further have a trophoblast layer. In some embodiments, the compositions disclosed herein can be a placenta graft.

In some embodiments, the compositions disclosed herein can be lyophilized, dehydrated, or micronized. In some aspects, the compositions disclosed herein can be first micronized and then lyophilized or dehydrated. In some aspects, the compositions disclosed herein can be first dehydrated or lyophilized and then micronized.

Another aspect of the present disclosure provides for methods for forming any one of the compositions disclosed herein.

In one aspect, a method for forming any one of the compositions disclosed herein may comprise obtaining placental membrane tissue and removing or substantially removing the amniotic epithelium layer to achieve a modified amnion.

In some aspects, the method further comprises lightly scraping the amniotic epithelium layer, thereby removing or substantially removing the epithelium layer and forming a modified amnion.

In some aspects, the method further comprises contacting the amniotic epithelium layer with a cell lysis solution for a length of time, thereby removing or substantially removing the epithelial layer and forming a modified amnion.

In various aspects, the method comprises contacting the amniotic epithelium layer with a cell lysis solution for a length of time and lightly scraping the amniotic epithelium layer, thereby removing or substantially removing the epithelium layer and forming a modified amnion.

In any of the embodiments herein using a cell lysis solution, the cell lysis solution may be applied to the amniotic epithelium layer for about 1 to 30 minutes, from about 1 to 25 minutes, from about 1 to 20 minutes, from about 1 to 25 minutes, from about 1 to 10 minutes or from about 1 to 5 minutes. In some embodiments, the cell lysis solution is applied to the amniotic epithelium layer for about 1 minute, about 5 minutes, about 10 minutes, about 15 minutes, about 20 minutes or about 25 minutes.

In various aspects of the methods provided, the cell lysis solution may comprise a nonionic poloxyethylene surfactant. For example, the nonionic polyoxyethylene surfactant may comprise Triton X-100, Triton X-114, polyoxyethylene sorbitan monolaurate (Tween-20 or Tween-80), Nonidet P-40 (NP-40), Igepal® CA-630, Brij™ 35, or a combination of any thereof. In some embodiments, the nonionic polyoxyethylene surfactant comprises Nonidet P-40 (NP-40).

In various aspects, the cell lysis solution may further comprise a buffer and/or a salt. In some aspects, the buffer may comprise Tris-HCl, and/or the salt may comprise NaCl.

In any of the methods herein, the cell lysis solution and/or the placenta tissue may be at room temperature. Alternatively, the cell lysis solution and/or the placental tissue may be at a temperature of about 0 to 4 degrees Celsius (32-40 degrees Fahrenheit). Alternatively, the cell lysis solution and/or the placental tissue may be at body temperature (e.g., about 37 degrees Celsius). In some embodiments, the cell lysis solution and/or the placental tissue is from about 4 to 25 degrees Celsius, is about 25 to about 37 degrees Celsius or is about 37 to about 50 degrees Celsius.

Any of the methods provided herein may further comprise rinsing the modified amnion with a sterile saline solution thereby removing the cell lysis solution and cellular debris.

In various aspects, the methods provided herein may further comprise lyophilizing, dehydrating, and/or micronizing the composition.

In any of the methods or compositions herein the structure of the modified amnion, spongy intermediate layer, and chorion may remain intact, except for the removal of the epithelial cells from the amnion. In various aspects, the modified amnion, spongy intermediate layer, and chorion are not held together with suture or other mechanical mean.

In some embodiments, any one of the compositions disclosed herein can be decontaminated.

Another aspect of the present disclosure provides a kit containing one of the compositions disclosed herein. In some embodiments, the kit comprises one or more containers comprising any of the compositions provided herein. In other embodiments, the kit provides for the formation of any one of the compositions disclosed herein.

### BRIEF DESCRIPTION OF THE FIGURES

FIG. 1. Depicts a schematic of a human amniotic sac. The image depicts the basic sublayers of the amnion and chorion membranes the make up the amniotic sac.

FIG. 2 depicts representative Hematoxylin and Eosin (H&E) stained images of a native (FIG. 2A) or de-epithelialized (FIG. 2B) amnion-chorion membrane according to an exemplary method of the instant disclosure.

FIGS. 3A-3D depicts representative images showing an effect of excessive force in an exemplary method of de-epithelialization of an amnion layer from a placenta tissue. FIG. 3A shows untreated tissue. FIG. 3B shows placement of the cell scraper onto the epithelial layer of the amnion. FIG. 3C shows tissue damage as a result of forceful scraping. FIG. 3D shows more tissue damage (including extrusion of inner spongy layer) as a result of excessive forceful scraping.

FIGS. 4A-4B depicts representative Hematoxylin and Eosin (H&E) stained images of a native (FIG. 4A) or de-epithelialized (FIG. 4B) amnion-chorion membrane according to an exemplary method of the instant disclosure.

FIGS. 5A-5B depicts representative Hematoxylin and Eosin (H&E) stained images of a native (FIG. 5A) or de-epithelialized (FIG. 5B) amnion-chorion membrane according to an exemplary method of the instant disclosure.

FIG. 6 is a schematic of post-processing options (e.g., lyophilization, dehydration and/or micronization) available to de-epithelialized tissue according to methods of the instant disclosure.

### DETAILED DESCRIPTION OF THE INVENTION

Placental allografts can refer to membranes derived from the amniotic sac. The amniotic sac consists of two main membranes, the amnion and the chorion, which are separated by an intermediate (or spongy) layer. The amnion is the innermost membrane with an epithelial layer that lines the inside of the amniotic sac. The chorion is the outermost layer, facing outward toward the uterine environment. Both the amnion and chorion membranes are composed of a series of distinct tissue layers, each of which provides important physical and biological properties to the amniotic sac. The present disclosure is based in part on the surprising discovery by the inventors that de-epithelialization of the amnion layer is clinically useful. As described herein, de-epithelialization of the amnion layer can expose the extracellular matrix (basement membrane) for better cellular attachment and can create a membrane product that can be placed either “up” or “down” without the need for secondary adjustments (e.g., cutting) to fit to a target. These advantages improve the functionality, utility and convenience of placental membranes compared to currently available options in which the existence of the epithelial layer makes the product “sided”, requiring careful attention to product orientation when applying it to the treatment site. Also provided herein are methods of making a de-epithelialized amnion layer and medical uses of de-epithelialized amnion layers.

Unless otherwise required by context, singular terms as used herein and in the claims shall include pluralities and plural terms shall include the singular. For example, reference to “a protein” includes a plurality of such proteins and reference to “the protein” includes reference to one or more protein known to those skilled in the art, and so forth.

The use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

As used herein, placental allografts refer to membranes that are derived from the amniotic sac. The amniotic sac acts as a physical barrier between the subject and developing fetus, protecting the fetus throughout pregnancy. The amniotic sac can have a unique combination of strength and flexibility and possess biological characteristics that help to protect the fetus during pregnancy such as cloaking mechanisms that effectively hide the developing fetus from immune system of the pregnant subject. The placental membranes can also express antimicrobial factors and immunomodulatory cytokines that can prevent infections and inflammatory conditions within a uterine environment in addition to active growth factors that can support the rapidly growing and developing tissue. Compositions provided herein that can be derived from the amniotic sac can contain a modified amnion, a spongy intermediate layer, and a chorion.

In some embodiments, the amniotic sac disclosed herein can be harvested from a placenta. In some aspects, a placenta for use herein can be collected from a donor subject. In some aspects, a donor subject can be a mammalian subject, including but not limited to a human, a primate, artiodactyl, perissodactyl, cow, bison, horse, pig, goat, or the like. In some examples, a placenta is harvested from a human subject. In some aspects, a placenta can be harvested from a human during a full-term or near full-term Cesarean (C-section) birth. In some other aspects, a harvested placenta may be immediately processed for use as disclosed herein. In other aspects, a harvested placenta may be stored for later processing for use as disclosed herein. In some examples, a harvested placenta stored for later processing can be placed in a labeled, sterile container or bag and submerged in a suitable storage medium for later processing. In some examples, a suitable storage medium can include one or more components suitable for storing a harvested tissue. Non-limiting examples of such components include sodium chloride, phosphate, potassium, magnesium, calcium, dextrose, glucose, citrate, lactate, tris, HEPES, water (e.g., purified water, sterile water, or water for injection), Lactated Ringer's solution, Ringer's solution, phosphate-buffered saline (PBS), tris-buffered saline (TBS), Hank's balanced salt solution (HBSS), Dulbecco's phosphate-buffered saline (DPBS), Earle's balanced salt solution (EBSS), standard saline citrate (SSC), HEPES-buffered saline (HBS), Gey's balanced salt solution (GBSS), cell culture mediums (e.g., Delbecco's Modified Eagle Medium (DMEM), Minimum Essential Media (MEM), calcium chelators (e.g., EDTA) etc.

In some aspects, a placenta for use herein can be a functional placenta organoid. A functional placenta organoid for use herein can contain an amniotic sac or at least, the tissue structures encompassed in an amniotic sac. In some aspects, functional placenta organoid for use herein can be derived from a blastocyst, a trophoblast, a placental stem cell, and the like harvested from a mammalian subject. Methods of making placenta organoids for use herein are known in the art, at least in Turco et al., Nature 564, 263-267 (2018), the disclosures of which is hereby incorporated by reference in its entirety.

In some embodiments, a donor subject and/or donor tissue may be screened for at least one factor to determine if the harvested placenta is suitable for any of the uses described herein. In some aspects, a donor subject and/or donor tissue can be tested for one or more viruses or bacteria using serological tests, which can include without limitation antibody, nucleic acid, or culture testing. Non-limiting examples of viral and bacterial screening may include screening for the human immunodeficiency virus type 1 or type 2 (HIV-1 and HIV-2), the hepatitis B virus (HBV), the hepatitis C virus (HCV), human T-lymphotropic virus type I or type II (HTLV-1 and HTLV-II), CMV, Coronavirus, or Treponema pallidum (a bacterium that causes syphilis).

In some embodiments, a placenta for use herein can be processed by dissecting the membrane portion of a placenta from the placental disc and umbilical cord. A membrane portion of a placenta can be dissected away from the placental disc and umbilical cord using any method known to those of ordinary skill in the art. Non-limiting examples can include using a scalpel, a pair of surgical scissors, a rotary blade, etc. In some examples, a harvested placenta can be transferred to a surface suitable for dissection, such as a soft, nonporous mat, and the membrane portion dissected away from the rest of the placenta, e.g., using surgical scissors or a scalpel. The membrane portion dissected from the placenta for use herein can encompass an amnion, a spongy intermediate layer, and a chorion wherein the amnion, a spongy intermediate layer, and a chorion are unseparated.

The amnion can encompass an epithelial monolayer, a basement membrane, a compact layer, and a fibroblast layer. The epithelial layer of the amnion is composed of a single layer of epithelial cells that is in contact with the basement membrane of the amnion. In various aspects, the epithelial layer of the amnion may be removed to form a modified (de-epithelialized) amnion. Methods for removal of the epithelial layer of the amnion are described below. The basement membrane of the amnion is a thin layer comprising extracellular matrix components, including collagen types III, IV, and V, noncollagenous glycoproteins (e.g., laminins, fibronectins, and nidogens), and proteoglycans (e.g., perlecans). The compact layer of the amnion is a dense, fibrous network comprising extracellular matrix components, including collagens (e.g., collagen types I, III, V, and VI) and fibronectins and is almost devoid of cells. The fibroblast layer is the thickest layer of the amnion and comprises fibroblasts and extracellular matrix components, such as collagens (e.g., collagen types I, III, and VI) and noncollagenous glycoproteins (e.g., laminins, fibronectins, and nidogens). The amnion can encompass additional native cell types.

The spongy intermediate layer is the interface between the amnion and the chorion. The spongy intermediate layer includes extracellular matrix components, such as collagens (e.g., collagen types I, III, and IV), proteoglycans, and glycoproteins. The spongy intermediate layer can encompass additional native cell types.

The chorion is several times thicker than the amnion and is composed of three layers: a reticular layer, a basement membrane, and a trophoblast layer. The reticular layer is in contact with the intermediate layer and comprises extracellular components, such as collagens (e.g., collagen types I, III, IV, V, and VI) and proteoglycans. The basement membrane is between the reticular layer and trophoblast layer of the chorion. Components of the basement membrane of the chorion comprise collagens (e.g., collagen type IV), laminins, and fibronectins. The trophoblast layer comprises several layers of trophoblasts and is in contact with the maternal endometrium and is involved in immunomodulation and “cloaking” of the fetus. As used herein, the term “trophoblast layer” includes cells, extracellular matrix, or blood vessels that may be present and that are derived from the capsular decidua, the portion of the maternal endometrium facing the uterine cavity. The chorion can encompass additional native cell types.

In some embodiments, the viability of one or more cell types in the amnion of the presently disclosed composition is preserved. In some embodiments, the viability of one or more cell types in the chorion of the presently disclosed composition is preserved. In some embodiments, the viability of one or more cell types in the spongy intermediate layer of the presently disclosed composition is preserved. In embodiments, the viability of one or more cells types in the amnion, spongy intermediate layer, and/or chorion is preserved.

In some embodiments, the dissected membrane portion of the placenta can be cut into one or more sheets before, during, or after any step of the methods disclosed herein. As used herein, a “sheet” refers to any three-dimensional conformation that may be formed from the sheet, including but not limited to, a cylindrical shape (e.g., sleeve), a cone shape, etc. In some aspects, the dissected membrane portion of the placenta can be cut into about 1 sheet to about 50 sheets (e.g., about 1 sheet, about 2 sheets, about 3 sheets, about 4 sheets, about 5 sheets, about 6 sheets, about 7 sheets, about 8 sheets, about 9 sheets, about 10 sheets, about 15 sheets, about 20 sheets, about 25 sheets, about 30 sheets, about 40 sheets, about 50 sheets). In some aspects, the dissected membrane portion of the placenta can be cut into any shape or size sheet that the tissue may accommodate. In some aspects, the dissected membrane portion of the placenta can be cut to have any surface area that the tissue may accommodate, including a surface area of about 1 mm2 to about 50 dm2 (e.g., about 1 mm2, about 5 mm2, about 1 cm2, about 5 cm2, about 10 cm2, about 25 cm2, about 50 cm2, about 75 cm2, about 1 dm2, about 25 dm2, about 50 dm2).

In some embodiments, the dissected membrane portion of the placenta can be de-epithelialized. As used herein, the term “de-epithelialized” refers to the process of removing some or all of the epithelial layer of the amnion. The dissected membrane portion of the placenta can be de-epithelialized to remove or substantially remove the epithelial layer of the amnion. In some aspects, the dissected membrane portion of the placenta can be de-epithelialized to remove 100% of the epithelial layer of the amnion. In some aspects, the dissected membrane portion of the placenta can be de-epithelialized to substantially remove the epithelial layer of the amnion. “Substantially” remove may mean that less than 5% of the epithelial layer of the amnion remains (i.e. 95% of the epithelial layer has been removed). In some other aspects, the dissected membrane portion of the placenta can be de-epithelialized to remove some of the epithelial layer of the amnion. In some examples, the membrane can be de-epithelialized to remove at least 50% to at least 99.9% of the epithelial layer of the amnion. In some embodiments, the membrane may be de-epithelialized to remove at least 60% to at least 99.9% of the epithelial layer of the amnion. In other embodiments, the membrane may be de-epithelialized to remove at least 70% to at least 99.9% of the epithelial layer of the amnion. In some embodiments, the membrane may be de-epithelialized to remove at least 80% to at least 99.9% of the epithelial layer of the amnion. For example, the membrane may be de-epithelialized to remove at least 90% to at least 99.9% of the epithelial layer of the amnion. For example, the membrane can be de-epithelialized to remove at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9% of the epithelial layer of the amnion. In some embodiments, the dissected membrane portion of the placenta can be de-epithelialized to generate a de-epithelialized membrane with an exposed amnion basement membrane that is substantially free of epithelial cells.

In some embodiments, de-epithelialization can be performed on a dissected membrane portion of the placenta prepared as disclosed herein. In some embodiments, de-epithelialization can be performed on a sheet of dissected membrane portion of the placenta prepared as disclosed herein. De-epithelialization of the amnion can be performed by any method known to those of ordinary skill in the art. In some aspects, a method for preparing a de-epithelized membrane can comprise scraping the amnionic side of the membrane using, for example, a cell scraper, or other device containing a flat edge, to disrupt the epithelial layer until the surface becomes smooth.

In some aspects, a method for preparing a de-epithelized membrane can comprise contacting a cell lysis solution to the amnionic epithelial layer. This disrupts the cellular structure, thereby removing the epithelial layer.

In some aspects, a method for preparing a de-epithelized membrane can comprise contacting a cell lysis solution to the amnionic epithelial layer followed by physical scraping of the surface of the membrane with a cell scraper or other flat-edged object to remove the epithelial layer from the amnion.

In various aspects, the cell lysis solution comprises a mild detergent, such as a nonionic surfactant. For example, the cell lysis solution may comprise a nonionic poloxyethylene surfactant. Non-limiting surfactants that may be used in various aspects of the disclosure include Triton X-100, Triton X-114, polyoxyethylene sorbitan monolaurate (Tween-20 or Tween-80), Nonidet P-40 (NP-40), Igepal® CA-630, Brij™ 35, or a combination of any thereof. In various embodiments, the cell lysis solution comprises Nonidet P-40 (NP-40).

In various aspects, the cell lysis solution may comprise one or more other components to preserve the health of the tissue. For example, the cell lysis solution may comprise a buffer (e.g., Tris or HEPES) or a salt (e.g., NaCl).

In some aspects, the methods provided herein comprise contacting a cell lysis solution to the amnionic side of the membrane portion of the placenta. The cell lysis solution may contact the amnion for a length of time (e.g., from about 1 to 30 minutes). In some cases, the cell lysis solution contacts the amnion for about 1 to 30 minutes, about 1 to 25 minutes, about 1 to 20 minutes, about 1 to 15 minutes, about 1 to 10 minutes or about 1 to 5 minutes. In some instances, the cell lysis solution contacts the amnion for about 5 minutes, about 10 minutes, about 15 minutes, about 20 minutes or about 25 minutes. In some cases, the cell lysis solution contacts the amnion for about 20 minutes.

In any of the methods herein, the placental tissue and/or cell lysis solution may be at a refrigerated temperature (e.g., from about 0 to 4 degrees Celsius). In some aspects, the placental tissue and/or cell lysis solution may be at room temperature (e.g., about 20-25 degrees Celsius). In some aspects, the placental tissue and/or cell lysis solution may be between refrigerated temperature and room temperature (e.g., from about 4 to 25 degrees Celsius). In some other aspects, the placental tissue and/or cell lysis solution may be at body temperature (e.g., at about 37 degrees Celsius). In some aspects, the placental tissue and/or cell lysis solution may be between room temperature and body temperature (e.g., from about 25 degrees Celsius to about 37 degrees Celsius). In some embodiments, the placental tissue and/or cell lysis solution may be at a temperature higher than body temperature (e.g., from about 37 to 50 degrees Celsius). In various aspects, the amount of time the cell lysis solution is contacted to the amnion can be adjusted based on the temperature of the cell lysis solution and/or the placental tissue (e.g., a higher temperature, lower exposure time).

Accordingly, in various aspects, a method for preparing a composition provided herein (e.g., preparing a de-epithelialized amniotic membrane) comprises scraping the amnion layer to remove or substantially remove the epithelial cell layer.

In other aspects, a method for preparing a composition provided herein comprises applying a cell lysis solution to the amnion, thus removing or substantially removing the epithelial cell layer.

In still other aspects, a method for preparing a de-epithelized membrane can comprise both applying the cell lysis solution and scraping the amnion layer in various combinations. For example, the method can comprise applying a cell lysis solution to the amnion, waiting a period of time (e.g., 20 minutes) and then lightly scraping the epithelial cell layer. In other aspects, the method can comprise lightly scraping the epithelial cell layer, then applying a cell lysis solution to the amnion and waiting a period of time (e.g., 20 minutes). In some instances, the epithelial cell layer may be scrapped again, after incubating with the cell lysis solution. In some aspects, one or more rounds of scraping and cell lysing may be utilized (starting with either the scraping or cell lysing step). In any of these methods, the cell lysis solution and any cellular debris may be rinsed from the tissue with a sterile saline solution (e.g., before scraping or after scraping).

In some aspects, the dissected membrane portion of the placenta can be placed chorion layer side down during the de-epithelialization process. In some other aspects, the de-epithelialization process can be monitored by visualizing the amnion side of the dissected membrane with a dissection microscope or any other form of magnification or sample preparation that would always the visualization of membrane surface. In some examples, the de-epithelialization process is repeated at least once until there is no visualization of an epithelial cell layer on the amnion. In some other examples, the de-epithelialization process is repeated at least once until there is visualization of at least 50% to at least 99.9% epithelial cell layer on the amnion. In some aspects, the de-epithelialization process can conclude once any damage of the underlying tissues is observed with a dissection scope. In some aspects, visualization of damage to the underlying tissues during the de-epithelialization process can be used as a factor to determine the amount of force used in removing the epithelial cell layer with a cell scraper or flat object as disclosed herein. In some examples, visualization of damage to the underlying tissues during the de-epithelialization process can indicate need of less force during the scraping process.

In some embodiments, the de-epithelialized membrane portion of a placenta can include an intact basement membrane. An intact basement membrane as used herein can include a modified amnion, a spongy intermediate layer, and a chorion, wherein the modified amnion does not contain an amniotic epithelium layer and wherein the spongy intermediate layer is disposed between the modified amnion and the chorion. In some aspects, an intact basement membrane herein can provide environmental cues to cells. In some aspects, environmental cues can aid in cell orientation, cellular response, cell growth and the like. In some aspects, environmental cues modify protein expression, gene expression, receptor-ligand binding, cellular signaling cascades and the like. In some aspects, environmental cues can include, but are not limited to cytokines, extracellular matrix components, growth factors, and hormones. For example, the binding of soluble growth factors to growth factor receptors and/or a cell's attachment to the extracellular matrix through integrins, which are also embedded in the cell membrane, can activate cellular signaling cascades. Integrins do not bind soluble factors; instead, they bind to the extracellular matrix (ECM). When bound to the ECM, integrins can also undergo conformational changes that result in the activation of signaling inside the cell. The downstream signaling of integrin-mediated pathways feeds into the complex web of signals being received from the external environment, contributing to the final messages that converge in the nucleus. The internal domains of integrins can also attach to the cell's cytoskeleton, allowing the cell to use the attachment of integrins to the ECM to generate the mechanical forces that enable cellular spreading and migration. Integrin receptors are formed by the dimerization of alpha and beta subunits. With 18 known alpha isoforms and 8 known beta isoforms, there are over 100 unique integrin pairs—any of which could apply to the present disclosure. Each alpha-beta dimer creates a specific binding pocket, providing integrins with tremendous versatility in what they will and will not bind to in the ECM. As an example, but not limited to, one set of alpha-beta combinations will bind specifically to fibrinogen, whereas a different set of pairing will bind to laminins, and yet another set of pairings will bind to collagens. Accordingly, signaling from distinct pairs can create a different signaling pattern on the inside of the cell. This idea becomes extremely important when considering the biological advantage of the de-epithelialized amnion-chorion membranes described. Cells are uniquely tuned to identify the extracellular matrix proteins of basement membranes as a way of orienting themselves within the body. This becomes particularly important in environments where basement membranes are often damaged or missing altogether.

In some embodiments, a de-epithelialized membrane portion of a placenta prepared as described herein can be washed at least once. As used herein, the term “wash” refers to any method suitable for removing any material (e.g., blood, tissues, cellular debris) from the de-epithelialized membrane in a suitable washing solution. Non-limiting examples of washing methods for use herein can include flushing, immersing, perfusing, soaking, or agitating in the presence or absence of pressure or vacuum. In some embodiments, the agitating is performed using a rocker, shaker, stir plate, rotating mixer, or other equipment capable of agitating. In some aspects, a membrane portion of a placenta can be washed at least once before the de-epithelialization process. In some other aspects, membrane portion of a placenta can be washed at least once during the de-epithelialization process. In still some other aspects, membrane portion of a placenta can be washed at least once after the de-epithelialization process. In any of these embodiments, the membrane portion of the placenta may be washed with a sterile saline solution.

In some embodiments, de-epithelialized membranes disclosed herein are prepared in a manner to increase the retention of endogenous soluble factors. Non-limiting examples of soluble factors can include antimicrobial peptides, growth factors, cytokines, or a combination thereof. In various embodiments, methods for preparing de-epithelialized membranes disclosed herein use wash steps wherein no salt is used. In some embodiments, one or more ionic and/or non-ionic detergent may be used. In various embodiments, methods for preparing de-epithelialized membranes disclosed herein use wash steps wherein the washing buffer is a low ionic strength buffers to retain at least one endogenous soluble factor. The buffers of the present disclosure are characterized herein using the term “ionic strength”. The term “ionic strength” as used herein is a dimensionless number defined by the equation: Ionic strength=0.5Σ(CiZi2), where Ci is the molar concentration of ionic species i, and Zi is the valence of ionic species i. In some aspects, a low ionic strength buffer used herein can have an ionic strength of at least 0.01 (e.g., about 0.01 to about 0.13). In some aspects, a low ionic strength buffer used herein can have a pH that is about the pH of the native tissue (e.g., harvested placenta). In some examples, a low ionic strength buffer used herein can have a pH ranging from about 6.4 to about 8.4. In some other example, a low ionic strength buffer used herein can have a pH of about 7.4. In some aspects, a low ionic strength buffer used herein can have less than about 20% salt, wherein a “salt” can be any chemical compound consisting of an ionic assembly of cations and anions. In some other aspects, a low ionic strength buffer used herein can have about 1% to less than about 20% (e.g., less than about 15%, less than about 10%, or less than about 5%) salt. In still some other aspects, a low ionic strength buffer used herein can have about 1% to less than about 20% (e.g., less than about 15%, less than about 10%, or less than about 5%) potassium chloride (KCl). In yet some other aspects, a low ionic strength buffer used herein can have about 1% to less than about 20% (e.g., less than about 15%, less than about 10%, or less than about 5%) sodium chloride (NaCl). In some aspects, a higher ionic strength buffer may be used. For example, a buffer comprising greater than 20% of a salt (e.g., KCl or NaCl) may be used in some embodiments.

In some embodiments, methods of de-epithelialization of a membrane portion of a placenta do not disrupt the structure of the amnion, spongy intermediate layer, and chorion other than removing the epithelial cells from the amnion to produce a modified amnion. In some embodiments, methods of de-epithelialization can result in modified amnion, spongy intermediate layer, and chorion that are not held together with suture or other mechanical means. In some embodiments, methods of de-epithelialization can result in modified amnion, spongy intermediate layer, and chorion, wherein one or more sections or layers have not been separated and reassembled. In some embodiments, methods of de-epithelialization can result in modified amnion, spongy intermediate layer, and chorion, wherein none of the sections or layers have been separated and reassembled.

In some embodiments, a de-epithelialized membrane portion of a placenta can be used immediately for any of its uses disclosed herein. In some embodiments, a de-epithelialized membrane portion of a placenta can be prepared for storage until later use. Any de-epithelialized membrane described herein can be decontaminated before, during, or after processing, including after final packaging. Decontamination may be performed using any methods known to one of skill in the art, including but not limited exposure to gamma radiation, E-beam radiation, ethylene oxide with a stabilizing gas (such as carbon dioxide or hydrochlorofluorocarbons (HCFC)), peracetic acid, hydrogen peroxide gas plasma, or ozone.

In some embodiments, a de-epithelialized membrane described herein can be stored in a suitable preservation medium at a suitable temperature for a suitable amount of time. Non-limiting examples of a suitable preservation medium can include water, a buffer, a saline solution, petrolatum, petroleum jelly, Vaseline, soft paraffin, glycerol, or Ringer's solution. In some aspects, a de-epithelialized membrane described herein can be stored at room temperature or can be refrigerated in a suitable preservation medium for a specific amount of time. In some examples, a de-epithelialized membrane described herein can be stored at a temperature of about 1° C. to about 30° C. for about 6 hours to about 84 hours (e.g., about 6 hours, about 12 hours, about 24 hours, about 48 hours, about 60 hours, about 72 hours, about 84 hours).

In some embodiments, a de-epithelialized membrane described herein does not require lamination prior to storage. In some embodiments, a de-epithelialized membrane described herein is a non-laminate membrane.

In some embodiments, a de-epithelialized membrane described herein can be preserved by cryopreservation, refrigeration, freezing, or dehydration. Once preserved, the de-epithelialized membrane may not require storage in a preservation medium. In some aspects, a de-epithelialized membrane described herein can be cryopreserved by freezing at e.g., liquid nitrogen or dry ice temperature, or a temperature of about −200° C. to about −40° C. and storing at liquid nitrogen temperatures for up to about 5 years. In some examples, a cryopreserved de-epithelialized membrane described herein can be thawed at least 24 prior to use.

In some aspects, a de-epithelialized membrane described herein can be dehydrated by any methods known in the art. In some examples, a de-epithelialized membrane described herein can be heat-dehydrated. A de-epithelialized membrane to be heat-dehydrated can be laid onto a flat drying surface that can be placed into a vacuum drying oven at suitable drying temperature and vacuum pressure. By controlling the drying temperature and vacuum pressure, it is possible to slowly remove the water from the membrane without disrupting the biological utility of the tissue. In some examples, a drying temperature and vacuum pressure suitable for use herein can be at least about 100° C. and at least about 0.01 mmHg, respectively.

In some aspects, a de-epithelialized membrane described herein can be dehydrated by chemical dehydration, for example by using a dehydration fluid that decreases the water content of the product. A dehydration fluid may be a fluid comprising an alcohol, an organic solvent, a hydrophilic polymer (e.g., polyoxyethylene oxide), a polysaccharide (such as a cellulose derivative or dextrose, etc.), or a salt.

In some aspects, a de-epithelialized membrane described herein can be dehydrated by lyophilization. Any method of lyophilization known to one of skill in the art is suitable for use herein. In some embodiments, a de-epithelialized membrane may be frozen and then lyophilized. For example, a de-epithelialized membrane can be quickly frozen rapidly by submersion in liquid nitrogen before lyophilizing or frozen less rapidly by other mechanisms before lyophilizing. In some embodiments, a de-epithelialized membrane can be stored for a period of time at a freezing temperature before lyophilization, such as for about 5 minutes to about 84 hours. In some aspects, the de-epithelialized membrane can be stored at ultra-low temperatures (e.g., −70 degrees Celsius or lower) for an extended period of time (e.g., days, weeks, or years) before lyophilization. In some aspects, a de-epithelialized membrane to be lyophilized (freeze-dried) can be laid onto a flat surface that can be frozen under controlled conditions that will not lead to the formation of water crystals within the membrane that would significantly disrupt the structural network of the tissue. In some examples, at least one cryoprotectant can be soaked into the de-epithelialized membrane before the freezing process is initiated in order to prevent the formation of water crystals in the membrane that would significantly disrupt the structural network of the tissue. Non-limiting examples of cryoprotectants can include glycerol, dimethyl sulfoxide (DMSO), and polyethylene glycol (PEG). In some examples, some additives, such as macromolecules and sugars, may be added to further decrease the damages on cells and tissues during cryopreservation. Once frozen to a desired temperature, the membrane is placed into a lyophilizer (vacuum chamber with attached cooling coils to capture sublimated water), where the water is removed under vacuum pressures that allow the sublimation of water from the tissue without significantly disrupting the structural network of the tissue.

In some embodiments, a dehydrated de-epithelialized membrane described herein can be further processed into a desired shape at any desired dimension. Defined dimensions can include, but are not limited to, two-dimensional sheet formats such as 8 mm×8 mm, 10 mm×10 mm, 20 mm×20 mm, 10 mm×10 mm, 15 mm×30 mm, or any other set of dimensions that would be of use for clinical applications. Sheet formats would not be confined to square or rectangular shapes as these membranes could be cut into any number of shapes (circles, ovals, triangles, pentagons, hexagons, etc.). In some examples, a dehydrated de-epithelialized membrane described herein can be “stamped” or have an impression placed into the shape as desired.

In some embodiments, a de-epithelialized membrane described herein can be further processed by micronization. As used herein, “micronization” refers to the dispersion of a de-epithelialized membrane into particles. Any methods of micronization known in the art is suitable for use herein. In some aspects, micronization of a de-epithelialized membrane can result in a protein particle dispersion encompassing a defined particle size distribution while substantially retaining the protein activity. In some aspects, micronization of a de-epithelialized membrane can result in a particle dispersion suitable for use in injectable pharmaceutical formulations. In some aspects, a de-epithelialized membrane can be first micronized and then lyophilized or dehydrated. In some other aspects, a de-epithelialized membrane can be first dehydrated or lyophilized and then micronized.

In various embodiments, de-epithelialized membranes described herein can be used in a manner that does not require a specific orientation during application. The de-epithelialization of the amnion layer provides for significant handling benefits when compared to non-de-epithelialized amnion-chorion membrane products. Removal of the epithelium and exposure of the underlying intact basement membrane creates a composition that does not require specific orientation in or on, for example but not limited to, a wound, to function successfully. Epithelial layers create a non-adhesive surface that prevents cellular attachment, requiring that other compositions having epithelial layers be placed with specific orientation. Failure to place these membranes having epithelial layers with the designated orientation (“up” or “down”) can affect product function and treatment outcomes, forcing these membranes to include markers on the membranes (e.g. embossed logos). The removal of the epithelial layer using methods described herein allows the membrane to be placed either “up” or “down” at the treatment site, eliminating the need for clinicians to focus their attention on this detail in the middle of their procedure.

In various embodiments, de-epithelialized membranes described herein do not require special handling prior to use at a treatment site. A handling advantage of removing the epithelial cell layer of the amnion as disclosed herein is the ability to place these membranes without having to cut them to fit the treatment site. In some aspects, de-epithelialized membranes described herein can be folded for use at a treatment site. The fact that these membranes can fold over on themselves without creating issues related to membrane orientation, eliminates the risk of creating localized areas in which the orientation of the membrane is incorrect because a corner or edge has folded under the membrane or back onto itself.

Any of the de-epithelialized membranes disclosed herein can be used for therapeutic, diagnostic, and/or research purposes, all of which are within the scope of the present disclosure. In various embodiments, de-epithelialized membranes described herein can have one or more medical uses. In some aspects, de-epithelialized membranes described herein can be used as a placental graft. The unique physical properties of placental allograft membranes for use herein can result from the complex extracellular matrix compositions of the amnion and chorion sublayers. Whereas the basement membranes provide barrier function and support for cellular layers, the compact and reticular layers provide the elastic and tensile strength that allows the membrane to stretch and bend without failing. The fibroblast in trophoblast layers do not contribute significantly to physical strength of the membrane; instead, these layers contain most of the soluble factors that are attributed to these membranes, including antimicrobial peptides, growth factors, and cytokines. In some examples, de-epithelialized membranes disclosed herein can have the physical properties of placental allograft membrane. In some examples, de-epithelialized membranes disclosed herein contain at least one soluble factor. In some examples, de-epithelialized membranes disclosed herein contain antimicrobial peptides, growth factors, cytokines, ECM components, cells, or a combination thereof.

In various embodiments, de-epithelialized membranes described herein can be used as wound covers. Placental membranes, as natural barrier membranes, are uniquely suited for wound healing environments. Placed as a protective cover over wounds, de-epithelialized membranes described herein can serve as a physical barrier that prevents pathogens and debris from entering the wound environment. De-epithelialized membranes used as wound covers can also have antimicrobial factors that can limit the reestablishment of infections that would cause a delay healing.

In some aspects, a wound suitable for treatment herein can be a socket resulting from the removal of one or more teeth from a subject. The jawbone has a natural tendency to become narrow and lose its original shape because the bone quickly resorbs after tooth removal. Bone loss can compromise the ability to place a dental implant (to replace the tooth) or its aesthetics and functional ability. Socket preservation procedures attempt to prevent bone loss by bone grafting the socket immediately after tooth extraction. With the procedure, the gum is retracted, the tooth is removed, and a void-filling material (usually a bone substitute) is placed in the tooth socket. A barrier membrane then is placed over the socket and graft material using a sterile forceps, and the gums are sutured closed over the membrane. In procedures like these, but not limited to, a de-epithelialized membrane described herein can serve as a wound cover to prevent loss of the bone graft material as well as separate the extraction socket from the oral cavity. In some examples, the membrane can protect the underlying tissue during the early stages of wound healing and tissue repair/regeneration and blocks unwanted infiltration of fibrotic gingival tissues (gums) into the depth of the socket during wound closure. The exposure of the basement membrane by de-epithelialization facilitates the ability of gingival cells to bind to the allograft membrane and begin the process of rebuilding/reestablishing a new gingival layer over the socket.

In various embodiments, de-epithelialized membranes described herein can be used as tissue barriers. In some aspects, de-epithelialized membranes described herein can be used in wound healing environments with partial or no exposure to an external environment. Often in surgical procedures on a subject, it is useful to separate two distinct tissue types to prevent tissues from growing together. Non-adhesive membrane products, or adhesion barriers, are commonly used in a wide range of surgical environments. While these can effectively separate distinct tissue types during wound healing, they do not provide any biological cues to direct cellular activities, including the reestablishment of natural tissue barriers. By exposing the basement membrane of the de-epithelialized membranes herein, such membranes can provide a natural barrier function as well as cues that can accelerate and guide the reestablishment of new tissues.

As an example, but not limited to, Guided Tissue Regeneration (GTR) refers to periodontal procedures attempting to regenerate or repair lost periodontal structures that provide structural support for teeth, including bone, periodontal ligaments, and cementum. Membrane products are employed in these procedures to separate distinct tissue types, protecting tissues that are slow to heal/regenerate (i.e. bone, periodontal ligaments, and cementum) from tissues that will, if allowed, aggressively invade the site (i.e. gingiva) and interfere with periodontal regeneration and repair. In these procedures, the de-epithelialized membranes described herein can create a natural tissue barrier, separating distinct periodontal tissue types and protecting slow-healing tissues during the early phases of tissue repair and regeneration. The exposure of the basement membrane facilitates the ability of gingival cells to bind to the allograft membrane and begin the process of rebuilding/reestablishing a new gingival layer over underlying tissues.

In some aspects, the de-epithelialized membranes herein may be micronized and/or formulated into a pharmaceutical form for use in treating a target disease in a patient. Suitable pharmaceutical forms may include a pharmaceutical composition comprising the de-epithelialized membranes and a pharmaceutically acceptable carrier (excipient). “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover, the disclosures of which is hereby incorporated by reference in its entirety. In some embodiments, the de-epithelialized membrane is micronized to form a powder which is then combined with an excipient for administration. Other pharmaceutical forms could include scaffolds or meshes that could be applied to a surface on a subject (e.g., a wound).

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover, the disclosures of which are hereby incorporated by reference in their entirety). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The pharmaceutical compositions to be used for in vivo administration must be sterile. This is readily accomplished by, for example, filtration through sterile filtration membranes. Therapeutic de-epithelialized membrane compositions are generally placed into a container having a sterile access port, for example, an intravenous solution bag or vial having a stopper pierceable by a hypodermic injection needle.

The pharmaceutical compositions described herein can be in unit dosage forms such as powders, granules, solutions or suspensions for parenteral (e.g., topical) or rectal administration. Other pharmaceutically acceptable forms can include mesh, matrix, or scaffolds or any other form suitable for wound healing and other uses known in the surgical arts.

In various embodiments, de-epithelialized membranes disclosed herein and pharmaceutical compositions containing such de-epithelialized membranes can be delivered to a subject in need thereof in an effective amount. The subject to be treated by the methods described herein can be a mammal, more preferably a human. Mammals include, but are not limited to, farm animals, sport animals, pets, primates, horses, dogs, cats, mice and rats. As used herein, “an effective amount” refers to the amount of each active agent required to confer therapeutic effect on the subject, either alone or in combination with one or more other active agents. Determination of whether an amount of the de-epithelialized membrane achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, de-epithelialized membranes that are compatible with the human immune system may be used to prolong half-life of the de-epithelialized membrane and to prevent the de-epithelialized membrane from being attacked by the host's immune system. Frequency of administration may be determined and adjusted over the course of therapy, and is generally, but not necessarily, based on treatment and/or suppression and/or amelioration and/or delay of a target disease/disorder.

In one example, dosages for a pharmaceutical composition comprising a de-epithelialized membrane as described herein may be determined empirically in individuals who have been given one or more administration(s) of the pharmaceutical compositions. To assess efficacy of the de-epithelialized membranes, an indicator of the disease/disorder can be followed.

The present disclosure also provides kits for use in treating or alleviating a target disease, such as wound healing as described herein. Such kits can include one or more containers comprising a de-epithelialized membrane, e.g., any of those described herein. In some instances, the de-epithelialized membrane may be co-used with a second therapeutic agent.

In some embodiments, the kit can comprise instructions for use in accordance with any of the methods described herein. The included instructions can comprise a description of administration (e.g., application) of the de-epithelialized membrane to a target (i.e., a wound), and optionally the second therapeutic agent, to treat, delay the onset, or alleviate a target disease (such as one linked to wound healing) as those described herein. The kit may further comprise a description of selecting an individual suitable for treatment based on identifying whether that individual has the target disease. In still other embodiments, the instructions comprise a description of administering a de-epithelialized membrane to an individual at risk of the target disease.

The instructions relating to the use of a de-epithelialized membrane can generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert can include indications that the composition is used for wound repair/healing.

The kits disclosed herein are in suitable packaging. Suitable packaging includes, but is not limited to, vials, bottles, jars, flexible packaging (e.g., sealed Mylar or plastic bags), and the like. Also contemplated are packages for use in combination with a specific device, such as a scaffold, a mesh, a graft, or other surgical device. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above. Kit can contain at least one or more buffers suitable for rehydration of a dehydrated de-epithelialized membrane. Kits can also contain instructions detailing how to reconstitute the dehydrated de-epithelialized membranes of the present disclosure.

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. 1. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.), the disclosures of which are hereby incorporated by reference in their entirety.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Tissue Procurement and Processing—Protocol A

Placental allograft tissues were procured using strict guidelines put forth by the American Association of Tissue Banks (AATB) and the Food and Drug Administration (FDA). Potential donors are identified as healthy women undergoing elective cesarean sections at the end of full-term pregnancies. With full consent of the donors, placental tissues were collected at the time of child delivery, allowing the tissues to be collected and maintained within a sterile environment. All donated tissues were thoroughly screened for microbiological and viral pathogens in accordance with AATB and FDA guidelines.

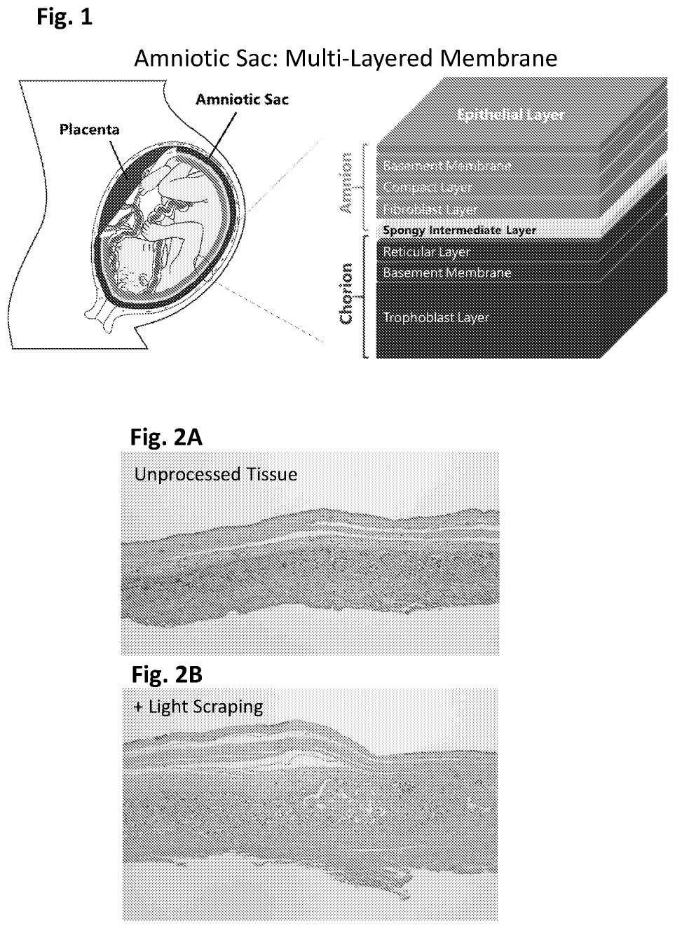

Following procurement in a sterile environment and transfer to a processing facility, the amniotic sac underwent a gentle washing and rinsing with solutions designed to maintain the unique biological properties of these membranes. As shown in FIG. 1, the amniotic sac consists of two main membranes, the amnion and the chorion. The amnion is the innermost membrane with an epithelial layer that lines the inside of the amniotic sac. The chorion is the outermost layer, facing outward toward the uterine environment. Both the amnion and chorion membranes are composed of a series of distinct tissue layers, each of which provides important physical and biological properties to the amniotic sac.

To remove the epithelial layer, the amnion-chorion membrane was placed into processing tray with the chorion layer down. A cell scraper, or other device containing a flat edge, was then used to carefully scrape the top of the membrane and disrupt the delicate epithelial layer until the surface becomes smooth.

Selective decellularization of the epithelial layer of the amnion can be observed in the histological sections presented in FIG. 2. FIG. 2A shows a cross section of a native amnion-chorion membrane stained with Hematoxylin and Eosin (H&E). In this image, the epithelial layer is clearly observed on the amnion as darkly stained cells lining the top of the tissue. In FIG. 2B, a cross section of the de-epithelialized amnion-chorion membrane stained with H&E is shown. In this image, the darkly stained cells that normally line the top of the membrane tissue are reduced, indicating that light scraping has removed at least a portion of the epithelial layer.

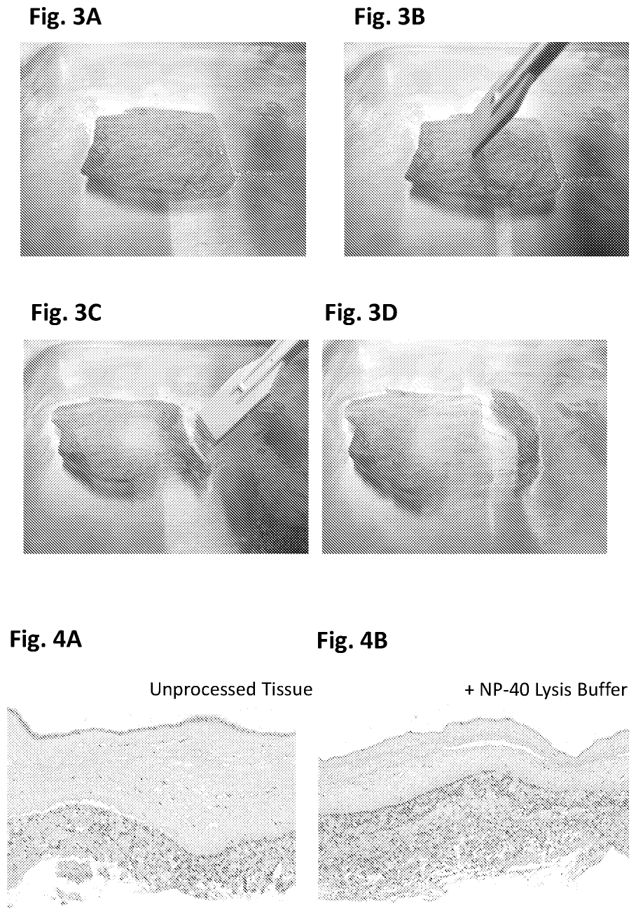

It was observed that excessive downward force when scraping the amnion surface resulted in squeezing out the bulk of underlying tissue. This was shown in FIG. 3 which illustrates untreated membrane (FIG. 3A); placement of the cell scraper on the epithelial layer of the amnion (FIG. 3B); scraping with force leads to tissue damage (FIG. 3C); and scraping with force ultimately resulted in extrusion of the underlying soft tissue layers (FIG. 3D).

Accordingly, it was determined that force needed to be minimized as much as possible in a “scraping-only” protocol. Periodically, samples were examined microscopically to visualize the disappearance of the cellular layer and absence of damage to underlying tissues that can occur of excessive force is used when scraping the membrane. Once the membrane had been sufficiently de-epithelialized, the tissue is rinsed lightly to remove cellular debris and is prepared for preservation (Example 4).

### Example 2—Tissue Procurement and Processing—Protocol B

An alternative de-epithelialization protocol was performed employing a lysis buffer solution to disrupt the epithelial layer without physically damaging the sample. Placental membranes were obtained from a third-party company specializing in the procurement of donated birth tissues. Using strict guidelines put forth by the American Association of Tissue Banks (AATB) and the Food and Drug Administration (FDA), birth tissues were collected from consenting, healthy donors at the time of elective cesarean section procedures. The tissues were rinsed with a sterile saline solution (0.9% w/v sodium chloride) and then placed into sterile bags containing 250 ml saline solution. The collected tissue (placenta with attached amniotic sac and umbilical cord) was then shipped overnight on wet ice. All donated tissues were maintained in a sterile environment and were screened for microbiological and viral pathogens in accordance with AATB and FDA guidelines.

Upon arrival at the processing facility, the birth tissue was removed from the shipping solution and transferred to a tray within a sterile biosafety cabinet. The amniotic sac was then dissected away from the placenta and placed into a container holding 100 ml of 0.9% sterile saline, where it was gently washed to remove residual blood.

To remove the epithelial layer, the amnion-chorion membrane was placed into processing tray with the chorion layer down. The epithelial surface of the amnion layer was blotted gently to remove excess fluid, then a cell lysis buffer containing 50 mM Tris-HCl (pH 7.4), 150 mM NaCl, 1% NP-40, and 5 mM EDTA (Alfa Aesar, cat #J60766AK) was added dropwise until the entire epithelial layer had been covered. The lysis solution was allowed to sit on top of the membrane for 20 minutes before rinsing the membrane with sterile saline solution to remove the lysis buffer and cellular debris.

Selective decellularization of the epithelial layer of the amnion can be observed in the histological sections presented in FIG. 4. FIG. 4A shows a cross section of a native amnion-chorion membrane stained with Hematoxylin and Eosin (H&E). In this image, the epithelial layer is clearly observed on the amnion as darkly stained cells lining the top of the tissue. In FIG. 4B, a cross section of the de-epithelialized amnion-chorion membrane stained with H&E is shown. In this image, the darkly stained cells that normally line the top of the membrane tissue are mostly absent, indicating that treatment with the cell lysis buffer has effectively removed a majority of the epithelial layer. Of note, cells of the underlying layers of the amnion and chorion remain intact.

### Example 3—Tissue Procurement and Processing—Protocol C

A third de-epithelization protocol was conducted that combined treatment with a lysis buffer with light scraping to fully remove the epithelial layer. Placental membranes were obtained from a third-party company specializing in the procurement of donated birth tissues. Using strict guidelines put forth by the American Association of Tissue Banks (AATB) and the Food and Drug Administration (FDA), birth tissues were collected from consenting, healthy donors at the time of elective cesarean section procedures. The tissues were rinsed with a sterile saline solution (0.9% w/v sodium chloride) and then placed into sterile bags containing 250 ml saline solution. The collected tissue (placenta with attached amniotic sac and umbilical cord) was then shipped overnight on wet ice. All donated tissues were maintained in a sterile environment and were screened for microbiological and viral pathogens in accordance with AATB and FDA guidelines.

Upon arrival at the processing facility, the birth tissue was removed from the shipping solution and transferred to a tray within a sterile biosafety cabinet. The amniotic sac was then dissected away from the placenta and placed into a container holding 100 ml of 0.9% sterile saline, where it was gently washed to remove residual blood.

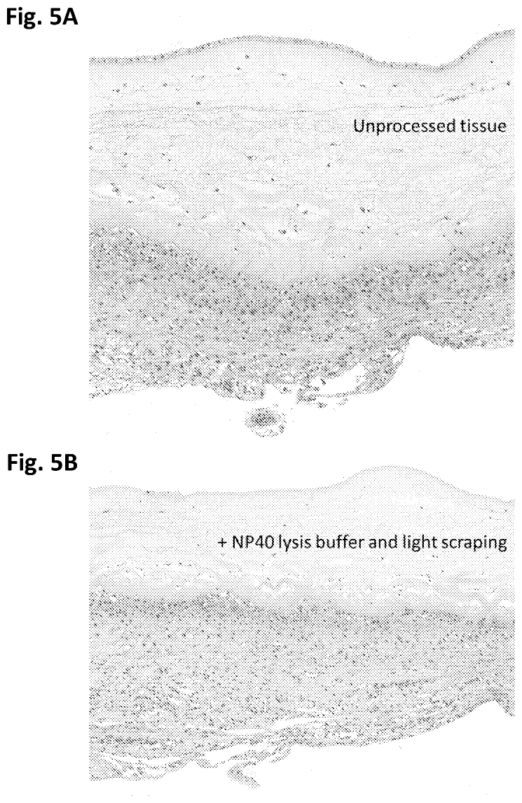

To remove the epithelial layer, the amnion-chorion membrane was placed into processing tray with the chorion layer down. The epithelial surface of the amnion layer was blotted gently to remove excess fluid, then a cell lysis buffer containing 50 mM Tris-HCl (pH 7.4), 150 mM NaCl, 1% NP-40, and 5 mM EDTA (Alfa Aesar, cat #J60766AK) was added dropwise until the entire epithelial layer had been covered. The lysis solution was allowed to sit on top of the membrane for 20 minutes before using a plastic cell scraper to lightly scrape the surface of the amnion layer. Care was taken to avoid applying excessive downward pressure while scraping to prevent damage to the sub-amnion layers that are composed of delicate connective tissues. Excessive downward force when scraping the amnion surface can result in squeezing out the bulk of underlying tissue as shown in FIG. 3. However, following application of the cell lysis solution, the epithelial layer could be removed with little force and minimal damage to underlying structures as shown in FIG. 5. Following application of cell lysis solution and incubation at room temperature for 10 minutes, light scraping and rinsing with sterile saline solution resulted in the facile removal of epithelial layer and cellular debris.

Selective decellularization of the epithelial layer of the amnion can be observed in the histological sections presented in FIG. 5. In FIG. 5A, a cross section of a native amnion-chorion membrane stained with Hematoxylin and Eosin (H&E) is shown. In this image, the epithelial layer is clearly observed on the amnion as darkly stained cells lining the top of the tissue. In FIG. 5B, a cross section of the de-epithelialized amnion-chorion membrane taken from the same tissue donation is shown (also stained with H&E). In this image, the darkly stained cells that normally line the top of the membrane tissue are mostly absent, indicating that treatment with the cell lysis buffer with light scraping has effectively removed a majority of the epithelial layer. Of note, cells of the underlying layers of the amnion and chorion remain intact.

### Example 4—Processing of De-Epithelialized Tissue

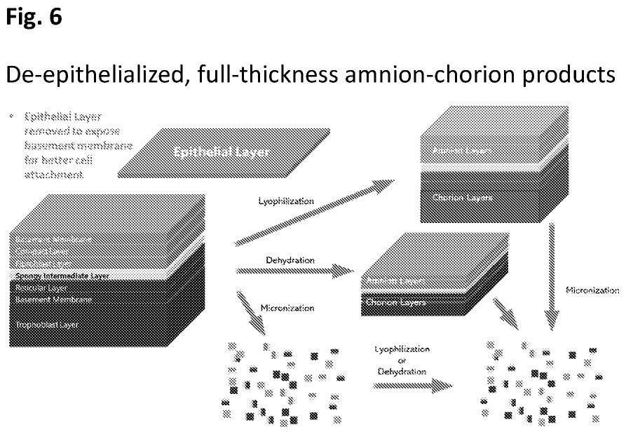

Following the de-epithelialization protocol of any of Examples 1-3, the membrane may be further processed for long term storage or use. Various processing options including lyophilization, dehydration, and micronization are shown in FIG. 6.

Following the de-epithelialization protocol, the membrane is then heat-dehydrated. In brief, it is laid onto a flat drying surface that can be placed into a vacuum drying oven. By controlling the drying temperature and vacuum pressure, it is possible to slowly remove the water from the membrane without disrupting the structural network of the tissue. Once all water is removed from the tissue, the heat-dehydrated membrane is processed into the desired shapes and sizes.

Alternatively, the membrane is dehydrated by lyophilization. In brief, the membrane is laid onto a flat surface that can be frozen under controlled conditions that do not lead to the formation of water crystals within the membrane. Formation of such water crystals can significantly disrupt the structural network of the tissue. Cryoprotectants are soaked into the membrane before the freezing process is initiated in order to prevent the formation of water crystals in the membrane that would significantly disrupt the structural network of the tissue. Once frozen to a desired temperature, the membrane is placed into a lyophilizer (vacuum chamber with attached cooling coils to capture sublimated water), where the water is removed under vacuum pressures that allow the sublimation of water from the tissue without significantly disrupting the structural network of the tissue. Once all water is removed from the tissue, the lyophilized membrane is processed into desired shapes and sizes.

Heat-dehydrated or lyophilized de-epithelialized amnion-chorion membranes are cut into defined dimensions and placed into individual packets, where they are terminally sterilized and packaged for distribution. Alternatively, the heat-dehydrated or lyophilized de-epithelialized amnion-chorion membranes are micronized according to standard procedures in the art (e.g., grinding, milling, or pulverizing) before being terminally sterilized and packaged for distribution.

We claim:

1. A composition comprising a modified amnion, a spongy intermediate layer, and a chorion, wherein the modified amnion comprises a first side which is an exposed basement membrane and wherein the spongy intermediate layer is disposed between the modified amnion and the chorion.

2. The composition of claim 1, wherein the exposed basement membrane is substantially free of epithelial cells.

3. The composition of claim 1 or 2, wherein the modified amnion further comprises a compact stromal layer and a fibroblast layer.

4. The composition of any one of claims 1 to 3, wherein the chorion comprises a basement membrane.

5. The composition of any one of claims 1 to 4, wherein the chorion further comprises a trophoblast layer.

6. The composition of any one of claims 1 to 5, wherein the composition is a placenta graft.

7. The composition of any one of claims 1 to 6, wherein the composition is lyophilized, dehydrated, micronized, or any combination thereof.

8. The composition of claim 7, wherein the composition is first micronized and then lyophilized or dehydrated.

9. The composition of claim 7, wherein the composition is first dehydrated or lyophilized and then micronized.

10. The composition of any one of claims 1 to 9, wherein the structure of the modified amnion, spongy intermediate layer, and chorion is intact, except for the removal of the epithelial cells from the amnion.

11. The composition of any one of claims 1 to 10, wherein the modified amnion, spongy intermediate layer, and chorion are not held together with suture or other mechanical means.

12. A method for forming a composition of any one of claims 1 to 11, wherein the method comprises: obtaining placental membrane tissue and removing or substantially removing the amniotic epithelium layer to achieve a modified amnion.

13. The method of claim 12, wherein the substantially removing the amniotic epithelium layer step comprises lightly scraping the amniotic epithelium layer, thereby removing or substantially removing the epithelium layer and forming a modified amnion.

14. The method of claim 12, wherein the substantially removing the amniotic epithelium layer step comprises contacting the amniotic epithelium layer with a cell lysis solution for a length of time, thereby removing or substantially removing the epithelial layer and forming a modified amnion.

15. The method of claim 12, wherein the substantially removing the amniotic epithelium layer step comprises contacting the amniotic epithelium layer with a cell lysis solution for a length of time and lightly scraping the amniotic epithelium layer, thereby removing or substantially removing the epithelium layer and forming a modified amnion.

16. The method of claim 14 or claim 15, wherein the length of time is from about 1 minute to about 30 minutes, from about 1 minute to about 25 minutes, from about 1 minute to about 20 minutes, from about 1 minute to about 15 minutes, from about 1 minute to about 10 minutes, or from about 1 minute to about 5 minutes.

17. The method of claim 16, wherein the length of time is about 1 minute, about 5 minutes, about 10 minutes, about 15 minutes, or about 20 minutes.

18. The method of any one of claims 14 to 17, wherein the cell lysis solution comprises a nonionic polyoxyethylene surfactant.

19. The method of claim 18, wherein the nonionic polyoxyethylene surfactant comprises Triton X-100, Triton X-114, polyoxyethylene sorbitan monolaurate (Tween-20 or Tween-80), Nonidet P-40 (NP-40), Igepal® CA-630, Brij™ 35, or a combination of any thereof.

20. The method of claim 19 wherein the nonionic polyoxyethylene surfactant comprises Nonidet P-40 (NP-40).

21. The method of any one of claims 14 to 20, wherein the cell lysis solution comprises a buffer and/or a salt.

22. The method of any one of claims 14 to 21, wherein the cell lysis solution and/or the placental tissue is at room temperature.

23. The method of any one of claims 14 to 21, wherein the cell lysis solution and/or the placental tissue is at a temperature of about 0 to 4 degrees Celsius (32 to 40 degrees Fahrenheit).

24. The method of any one of claims 14 to 21, wherein the cell lysis solution and/or the placental tissue is at body temperature (about 37 degrees Celsius).

25. The method of any one of claims 14 to 21, wherein the cell lysis solution and/or the placental tissue is from about 4 to 25 degrees Celsius, is about 25 to about 37 degrees Celsius or is about 37 to about 50 degrees Celsius.

26. The method of any one of claims 14 to 25, further comprising rinsing the modified amnion with a sterile saline solution thereby removing the cell lysis solution and cellular debris.

27. The method of any one of claims 12 to 26, wherein the method further comprises lyophilizing, dehydrating, and/or micronizing the composition.

28. The method of any one of claims 12 to 27, wherein the structure of the modified amnion, spongy intermediate layer, and chorion is intact, except for the removal of the epithelial cells from the amnion.

29. The method of any one of claims 12 to 28, wherein the modified amnion, spongy intermediate layer, and chorion are not held together with suture or other mechanical means.

30. A kit comprising one or more containers comprising a composition of any one of claims 1 to 11.

31. The composition of claim 1, wherein the modified amnion further comprises a compact stromal layer and a fibroblast layer.

32. The composition of claim 1, wherein the chorion comprises a basement membrane.

33. The composition of claim 32, wherein the chorion further comprises a trophoblast layer.

34. The composition of claim 1, wherein the composition is a placenta graft.

35. The composition of claim 1, wherein the composition is lyophilized, dehydrated, micronized, or any combination thereof.

36. The composition of claim 35, wherein the composition is first micronized and then lyophilized or dehydrated.

37. The composition of claim 35, wherein the composition is first dehydrated or lyophilized and then micronized.

38. The composition of claim 1, wherein the structure of the modified amnion, spongy intermediate layer, and chorion is intact, except for the removal of the epithelial cells from the amnion.

39. The composition of claim 1, wherein the modified amnion, spongy intermediate layer, and chorion are not held together with suture or other mechanical means.

40. A method for forming a composition of claim 1, wherein the method comprises: obtaining placental membrane tissue and removing or substantially removing the amniotic epithelium layer to achieve a modified amnion.

41. The method of claim 40, wherein the substantially removing the amniotic epithelium layer step comprises lightly scraping the amniotic epithelium layer, thereby removing or substantially removing the epithelium layer and forming a modified amnion.

42. The method of claim 41, further comprising rinsing the modified amnion with a sterile saline solution thereby removing the cell lysis solution and cellular debris.

43. The method of claim 41, wherein the method further comprises lyophilizing, dehydrating, and/or micronizing the composition.

44. The method of claim 41, wherein the structure of the modified amnion, spongy intermediate layer, and chorion is intact, except for the removal of the epithelial cells from the amnion.

45. The method of claim 41, wherein the modified amnion, spongy intermediate layer, and chorion are not held together with suture or other mechanical means.

46. The method of claim 40, wherein the substantially removing the amniotic epithelium layer step comprises contacting the amniotic epithelium layer with a cell lysis solution for a length of time, thereby removing or substantially removing the epithelial layer and forming a modified amnion.

47. The method of claim 46, wherein the length of time is from about 1 minute to about 30 minutes, from about 1 minute to about 25 minutes, from about 1 minute to about 20 minutes, from about 1 minute to about 15 minutes, from about 1 minute to about 10 minutes, or from about 1 minute to about 5 minutes.

48. The method of claim 47, wherein the length of time is about 1 minute, about 5 minutes, about 10 minutes, about 15 minutes, or about 20 minutes.

49. The method of claim 46, wherein the cell lysis solution comprises a nonionic polyoxyethylene surfactant.

50. The method of claim 49, wherein the nonionic polyoxyethylene surfactant comprises Triton X-100, Triton X-114, polyoxyethylene sorbitan monolaurate (Tween-20 or Tween-80), Nonidet P-40 (NP-40), Igepal® CA-630, Brij™ 35, or a combination of any thereof.

51. The method of claim 50 wherein the nonionic polyoxyethylene surfactant comprises Nonidet P-40 (NP-40).

52. The method of claim 46, wherein the cell lysis solution comprises a buffer and/or a salt.

53. The method of claim 46, wherein the cell lysis solution and/or the placental tissue is at room temperature.

54. The method of claim 46, wherein the cell lysis solution and/or the placental tissue is at a temperature of about 0 to 4 degrees Celsius (32 to 40 degrees Fahrenheit).

55. The method of claim 46, wherein the cell lysis solution and/or the placental tissue is at body temperature (about 37 degrees Celsius).

56. The method of claim 46, wherein the cell lysis solution and/or the placental tissue is from about 4 to 25 degrees Celsius, is about 25 to about 37 degrees Celsius or is about 37 to about 50 degrees Celsius.

57. The method of claim 46, further comprising rinsing the modified amnion with a sterile saline solution thereby removing the cell lysis solution and cellular debris.

58. The method of claim 46, wherein the method further comprises lyophilizing, dehydrating, and/or micronizing the composition.

59. The method of claim 46, wherein the structure of the modified amnion, spongy intermediate layer, and chorion is intact, except for the removal of the epithelial cells from the amnion.

60. The method of claim 46, wherein the modified amnion, spongy intermediate layer, and chorion are not held together with suture or other mechanical means.

61. The method of claim 40, wherein the substantially removing the amniotic epithelium layer step comprises contacting the amniotic epithelium layer with a cell lysis solution for a length of time and lightly scraping the amniotic epithelium layer, thereby removing or substantially removing the epithelium layer and forming a modified amnion.

62. The method of claim 61, wherein the length of time is from about 1 minute to about 30 minutes, from about 1 minute to about 25 minutes, from about 1 minute to about 20 minutes, from about 1 minute to about 15 minutes, from about 1 minute to about 10 minutes, or from about 1 minute to about 5 minutes.

63. The method of claim 62, wherein the length of time is about 1 minute, about 5 minutes, about 10 minutes, about 15 minutes, or about 20 minutes.

64. The method of claim 61, wherein the cell lysis solution comprises a nonionic polyoxyethylene surfactant.

65. The method of claim 64, wherein the nonionic polyoxyethylene surfactant comprises Triton X-100, Triton X-114, polyoxyethylene sorbitan monolaurate (Tween-20 or Tween-80), Nonidet P-40 (NP-40), Igepal® CA-630, Brij™ 35, or a combination of any thereof.

66. The method of claim 65 wherein the nonionic polyoxyethylene surfactant comprises Nonidet P-40 (NP-40).

67. The method of claim 61, wherein the cell lysis solution comprises a buffer, a salt, a calcium chelator, or any combination thereof.

68. The method of claim 61, wherein the cell lysis solution and/or the placental tissue is at room temperature.

69. The method of claim 61, wherein the cell lysis solution and/or the placental tissue is at a temperature of about 0 to 4 degrees Celsius (32 to 40 degrees Fahrenheit).

70. The method of claim 61, wherein the cell lysis solution and/or the placental tissue is at body temperature (about 37 degrees Celsius).

71. The method of claim 61, wherein the cell lysis solution and/or the placental tissue is from about 4 to 25 degrees Celsius, is about 25 to about 37 degrees Celsius or is about 37 to about 50 degrees Celsius.

72. The method of claim 61, further comprising rinsing the modified amnion with a sterile saline solution thereby removing the cell lysis solution and cellular debris.

73. The method of claim 61, wherein the method further comprises lyophilizing, dehydrating, and/or micronizing the composition.

74. The method of claim 61, wherein the structure of the modified amnion, spongy intermediate layer, and chorion is intact, except for the removal of the epithelial cells from the amnion.

75. The method of claim 61, wherein the modified amnion, spongy intermediate layer, and chorion are not held together with suture or other mechanical means.

76. A kit comprising one or more containers comprising a composition of claim 1.
